(19) United States
(12) Patent Application Publication
(10) Pub. No.: US 2022/0354423 A1
(43) Pub. Date: Nov. 10, 2022

(54) Force Sensor

(71) Applicant: Technische Universiteit Delft, Delft (NL)

(72) Inventor: Tim Horeman, Delft (NL)

(21) Appl. No.: 17/866,982

(22) Filed: Jul. 18, 2022

Related U.S. Application Data
(63) Continuation of application No. PCT/NL2020/050784, filed on Dec. 14, 2020.

(30) Foreign Application Priority Data
Jan. 17, 2020 (NL) 2024695

(51) Int. Cl. A61B 5/00; A61F 2/46; G01L 5/169; A61F 2/32

(52) U.S. Cl. CPC A61B 5/4851; A61F 2/4657; G01L 5/169; A61F 2/32; A61F 2002/4666

(57) ABSTRACT

A force sensor provided with a base and a table with legs mounted on the base. The table is movable with respect to the base by a force applied to the table, and the sensor is further provided with a measuring instrument or instruments for measuring a deflection of one or more of the legs, which deflection represents said force applied to the table.

### CROSS-REFERENCE TO RELATED APPLICATIONS

This application is a continuation of International Patent Application No. PCT/NL2020/050784, titled “Force Sensor”, filed on Dec. 14, 2020, which claims priority to and the benefit of Netherland Patent Application No. 2024695, titled “Force Sensor”, filed on Jan. 17, 2020, and the specification and claims thereof are incorporated herein by reference.

### BACKGROUND OF THE INVENTION

### Field of the Invention

The invention relates to a force sensor, particularly suitable for use in a temporary hip joint implant in order to measure the forces acting on the artificial hip joint and to use this information to improve the characteristics of a definite hip joint after implementation into the patient.

A problem with known force sensors that are particularly used for measuring the forces acting on an artificial hip joint is that strain gauges cannot be used in view of its requirement to be integrally construed with the hip joint, which hinders its application because cleaning and sterilization is virtually impossible without damaging the construction of the force sensor.

It is an object of the invention to provide a solution for this problem and to answer the need for a reliable force sensor with which the forces acting on an artificial hip joint can be measured. Eventually this leads to better solutions for the patient when he or she is provided with an artificial hip joint that is optimized in terms of its placement and its interference with the bones of the patient.

### Background Art

US-B1-10,486,314; US2006/0213287; CN-A-104568279; DE 10 2017 218 033 A1; US2014/0041461; and US2019/0025143 each disclose a force sensor which comprises a base, a table with legs mounted on the base and said table being movable with respect to the base by a force applied to the table, wherein the force sensor is further equipped with a measuring instrument or instruments for measuring a deflection of one or more of the legs, which deflection represents a measure of said force applied to the table.

Note that any reference to other references herein is given for more complete background and is not to be construed as an admission that such publications are prior art for patentability determination purposes.

### BRIEF SUMMARY OF THE INVENTION

A force sensor according to an embodiment of the present invention has the features of one or more of the appended claims.

Each leg of a force sensor according to an embodiment of the present invention is preferably connected to the base and is connected to the table, wherein there is a distance between the base and the table, and wherein each leg comprises a relatively thick portion and a relatively thin portion, wherein the relatively thick portion is fixed to the base and the relatively thin portion is fixed to the table, without the thick portion engaging the table and without the thin portion engaging the base, and wherein the thick portion and the thin portion are essentially parallel to each other, such that both the thick portion and the thin portion substantially span the distance between the base and the table, and that the thin portion comprises two parallel parts, a first part connected to the table and having a first extremity distant from the table, and a second part connected to the thick portion proximate to the table and provided with a second extremity distant from the table, which first extremity and second extremity merge into each other so as to arrange that a movement of the table with respect to the base that results in a corresponding movement of the first part of the thin portion that connects to the table, translates into an enlarged movement of the first extremity and second extremity that are merged into each other distant from the table.

To say it short, a relatively small movement of the table is thus amplified into a relatively large movement at the first and second extremities that are merged into each other distant from the table. This provides a very effective means for measuring the displacement of the table which is representative of the force that is applied to the table. Accordingly it is preferred that the measuring instrument or instruments are arranged to measure a displacement of the first extremity and second extremity that are merged into each other distant from the table.

Preferably the force sensor has three legs with which the table is mounted on the base, to provide the force sensor with three degrees of freedom for measuring.

Within the scope of the invention, several types of measuring instruments can be used in the force sensor. In one embodiment the measuring instrument or instruments comprise an optical displacement sensor or sensors. In another embodiment the measuring instrument or instruments comprise a Hall effect sensor or sensors for measuring a displacement of the first extremity and second extremity that are merged into each other distant from the table. In that embodiment it is preferred that the measuring instrument or instruments comprise a magnet or magnets mounted on the thin portion of the leg, preferably on the first extremity and/or second extremity that are merged into each other distant from the table.

One of the benefits of the force sensor according to an embodiment of the present invention is that it is possible to arrange that the measuring instrument or instruments are dismountable from the force sensor to enable cleaning and/or sterilization of the respective parts of the force sensor.

Another benefit of the force sensor according to an embodiment of the present invention is that it is not costly to manufacture, particularly when at least one of the base, the table and the legs is made from stainless steel.

In a suitable embodiment, the table connects to a ball of a hip joint implant. This opens the way to a use of the force sensor according to an embodiment of the present invention in a hip joint implant of a patient for measuring forces acting on the hip joint implant during movement of the patient.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is a three-dimensional illustration from a perspective view of a force sensor according to an embodiment of the present invention comprising a ball head;

FIG. 2 is an illustration from a perspective view showing an enlarged view of the force sensor of FIG. 1 with the ball head removed;

FIG. 3 shows a schematic drawing of particular mechanical parts of a force sensor according to an embodiment of the present invention;

FIGS. 4A and 4B are illustrations showing a 2D representation of a force sensor according to an embodiment of the present invention; and

FIGS. 5A and 5B are illustrations showing a representation of the movements of one of the legs of a force sensor according to an embodiment of the present invention.

### DETAILED DESCRIPTION OF THE INVENTION

Whenever in the figures the same reference numerals are applied, these numerals refer to the same parts.

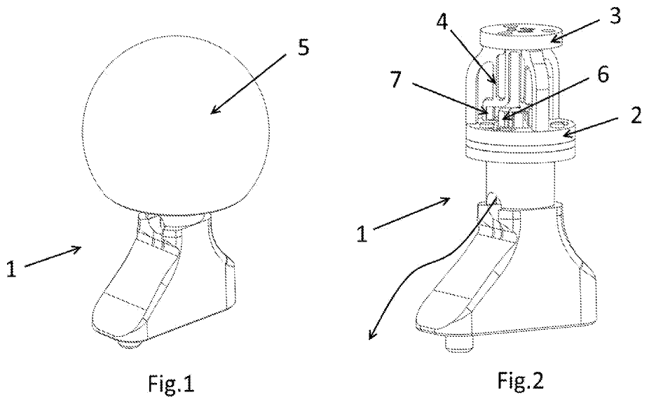

Making first reference to FIG. 2, particular details of a force sensor 1 according to an embodiment of the present invention are shown, notably base 2, table 3 with three legs 4 mounted on the base 2, wherein said table 3 is movable with respect to base 2 by a force F_{X}, F_{y}, F_{z} (see FIG. 1) applied to ball head 5 mounted on table 3. Force sensor 1 preferably has three degrees of freedom, for which purpose force sensor 1 is provided with the shown three legs 4 that support table 3 on base 2.

Further there is a measuring instrument or there are measuring instruments 6, 7 for measuring a deflection of one or more of legs 4, which deflection ultimately is representative for said force F_{X}, F_{Y}, F_{z} that is applied —indirectly- to table 3 through ball head 5. It is possible to apply optical instruments for the measurement of said displacement, but it is preferred to apply a Hall effect sensor or sensors 6 that cooperate with magnets 7 for measuring a displacement of legs 4. Magnets 7 are placed on legs 4, and Hall effect sensors 6 are stationary provided on base 2. To achieve best results magnets 7 are mounted on a thin portion 4″ of legs 4, preferably on first extremity 8′ and/or second extremity 9′ that are merged into each other distant from table 3 as will be discussed in the next paragraphs.

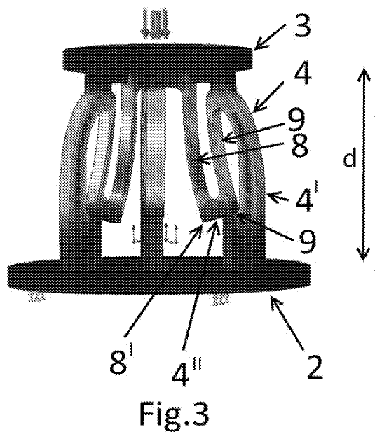

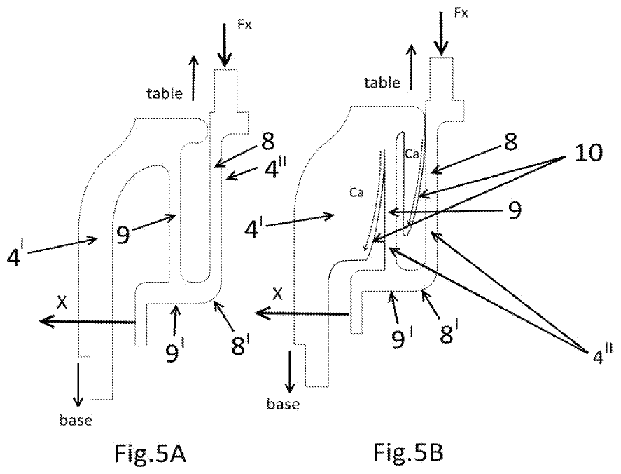

Specifically with reference to FIG. 3 and the detailed view provided by FIGS. 5A and 5B, the cooperation of notable parts of force sensor 1 of the invention can be explained.

FIG. 3 shows that each leg 4 is connected to base 2 and is connected to table 3, wherein there is a distance d between base 2 and table 3. FIGS. 5A and 5B both show one of the legs 4 separately from base 2 and table 3. Each leg 4 of the preferably three legs, comprises a relatively thick portion 4′ and a relatively thin portion 4″, wherein relatively thick portion 4′ is fixed to base 2 and relatively thin portion 4″ is fixed to table 3, without thick portion 4′ engaging table 3 and without thin portion 4″ engaging base 2. Thick portion 4′ and thin portion 4″ are essentially parallel to each other, such that both thick portion 4′ and thin portion 4″ substantially span the distance d between base 2 and table 3. Further, as may be best seen in FIGS. 5A and 5B, thin portion 4″ comprises two parallel parts, first part 8 connected to table 3 and having first extremity 8′ distant from table 3, and second part 9 connected to thick portion 4′ proximate to table 3 and provided with second extremity 9′ distant from table 3, wherein first extremity 8′ and second extremity 9′ merge into each other so as to arrange that a movement of table 3 with respect to base 2 that results in a corresponding movement of first part 8 of thin portion 4″ that connects to table 3, translates into an enlarged movement X of first extremity 8′ and second extremity 9′ that are merged into each other distant from table 3. It is at this location where first extremity 8′ and second extremity 9′ merge into each other, that the earlier mentioned magnet or magnets 7 are applied when the displacement measurement is carried out using a Hall sensor or sensors 6.

The difference between the construction of FIG. 5A and FIG. 5B is the following. FIG. 5A relates to an embodiment wherein the behaviour of leg 4 depends only on the material properties and its dimensions. FIG. 5B is embodied with particular curvatures 10 on thick portion 4′ of leg 4, which prevents high stress locations on weak locations of movable thin portion 4″ of leg 4, and accordingly increases the working range of force sensor 1.

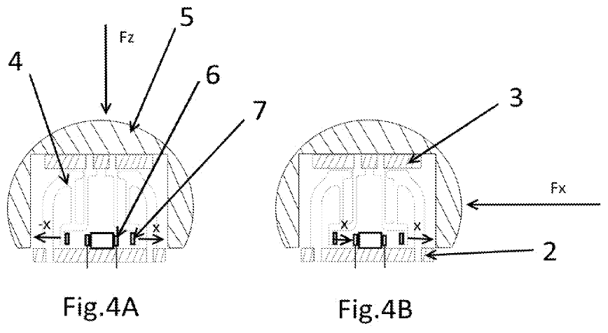

The elucidation of the previous paragraphs also applies to the 2D representation of FIGS. 4A and 4B depicting an indication how a force F_{z} in the Z-direction and a transversal force F_{X} that both may act on force sensor 1, cause a displacement of first extremity 8′ and second extremity 9′ that are merged into each other distant from table 3. The force F_{z} results in a symmetrical movement −X and X of the shown legs 4, whereas the transversal force F_{X} leads to an asymmetrical movement of legs 4. In these FIGS. 4A and 4B, it is also shown that for measuring the displacement of the merged first extremity 8′ and second extremity 9′, an optical sensor or a Hall effect sensor or sensors 6 can be applied. When use is made of a Hall effect sensor or sensors 6, it is preferred that magnets 7 are applied on first extremity 8′ and second extremity 9′ that are merged into each other distant from table 3. Preferably further, the measuring instrument or instruments are dismountable from force sensor 1 to enable cleaning and/or sterilization of the respective parts of force sensor 1.

It is further preferred that at least one of base 2, table 3 and legs 4 is made from stainless steel. This reduces manufacturing costs.

Preferably ball head 5 that connects to table 2 of force sensor 1 is a ball of a hip joint implant. Accordingly, force sensor 1 can suitably be used in a hip joint implant of a patient for measuring forces acting on the hip joint implant during movement of the patient.

Although the invention has been discussed in the foregoing with reference to an exemplary embodiment of the force sensor of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

1. A force sensor comprising:
a base;
a table with legs mounted on the base, and said table being movable with respect to the base by a force applied to the table; and
a measuring instrument or instruments for measuring a deflection of one or more of the legs, which deflection representing a measure of said force applied to the table,
wherein each leg is connected to the base and is connected to the table,
wherein there is a distance between the base and the table, and each leg comprises a relatively thick portion and a relatively thin portion,
wherein the relatively thick portion is fixed to the base and the relatively thin portion is fixed to the table, without the thick portion engaging the table and without the thin portion engaging the base, and wherein the thick portion and the thin portion are essentially parallel to each other, such that both the thick portion and the thin portion substantially span the distance between the base and the table, and the thin portion comprises two parallel parts, a first part connected to the table and having a first extremity distant from the table, and a second part connected to the thick portion proximate to the table and said second part having a second extremity distant from the table, which first extremity and second extremity merge into each other so as to arrange that a movement of the table with respect to the base that results in a corresponding movement of the first part of the thin portion that connects to the table, translates into an enlarged movement of the first extremity and second extremity that are merged into each other distant from the table.

2. The force sensor according to claim 1 comprising three legs with which the table is mounted on the base.

3. The force sensor according to claim 1, wherein the measuring instrument or instruments are arranged to measure a displacement of the first extremity and second extremity that are merged into each other distant from the table.

4. The force sensor according to claim 1, wherein the measuring instrument or instruments comprise an optical displacement sensor or sensors.

5. The force sensor according to claim 3, wherein the measuring instrument or instruments comprise a Hall effect sensor or sensors for measuring a displacement of the first extremity and second extremity that are merged into each other distant from the table.

6. The force sensor according to claim 5, wherein the measuring instrument or instruments comprise a magnet or magnets mounted on the thin portion of the leg, preferably on the first extremity and/or second extremity that are merged into each other distant from the table.

7. The force sensor according to claim 1, wherein the measuring instrument or instruments are dismountable from the force sensor to enable cleaning and/or sterilization of the respective parts of the force sensor.

8. The force sensor according to claim 1, wherein at least one of the base, the table and the legs comprises stainless steel.

9. The force sensor according to claim 1, wherein the table connects to a ball head of a hip joint implant.

10. The force sensor according to claim 2, wherein the measuring instrument or instruments are arranged to measure a displacement of the first extremity and second extremity that are merged into each other distant from the table.

11. The force sensor according to claim 2, wherein:
the measuring instrument or instruments are arranged to measure a displacement of the first extremity and second extremity that are merged into each other distant from the table;
the measuring instrument or instruments comprise an optical displacement sensor or sensors;
the measuring instrument or instruments comprise a Hall effect sensor or sensors for measuring a displacement of the first extremity and second extremity that are merged into each other distant from the table;
the measuring instrument or instruments comprise a magnet or magnets mounted on the thin portion of the leg, preferably on the first extremity and/or second extremity that are merged into each other distant from the table; and
the measuring instrument or instruments are dismountable from the force sensor to enable cleaning and/or sterilization of the respective parts of the force sensor.

12. The force sensor according to claim 11 wherein at least one of the base, the table and the legs comprises stainless steel.

13. The force sensor according to claim 11, wherein the table connects to a ball head of a hip joint implant.
